(19) United States
(12) Patent Application Publication
(10) Pub. No.: US 2008/0261227 A1
(43) Pub. Date: Oct. 23, 2008

(54) Kinetic Pcr Assay for Quantification of Gene Amplification on Chromosome 17

(75) Inventors: Lu Ping Shen, San Leandro, CA (US); Marc Munnes, Erkrath (DE); Ralph Markus Wirtz, Cologne (DE); Daryn Kenny, Pleasant Hill, CA (US); Charlene E. Bush-Donovan, Livermore, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NJ (US)

(21) Appl. No.: 12/093,603

(22) PCT Filed: Nov. 28, 2006
(86) PCT No.: PCT/US06/61288

§ 371 Date: May 14, 2008

Related U.S. Application Data
(60) Provisional application No. 60/740,307, filed on Nov. 28, 2005.

(51) Int. Cl. C12Q 1/68

(52) U.S. Cl. 435/6

(57) ABSTRACT

Provided is a kinetic PCR (“kPCR”) assay for determining gene copy number of a target gene located on chromosome 17. The kPCR assay uses the MMP-28 gene located at the 17q11.2-17q12 loci as a control and thus, is capable of detecting gene copy number of any gene on chromosome 17 in both singleplex and multiplex format without the need for a standard curve. The kPCR assay is useful for determining the gene copy number of the HER2/neu gene located at loci 17q12-17q21.32, which is a requirement for determining if a breast cancer patient is a candidate for anti-HER2/neu gene therapy.

### TECHNICAL FIELD

This invention relates generally to the detection and quantification of genes on chromosome 17. More specifically, the invention relates to the detection of the HER2/neu gene, which is located on the long arm of chromosome 17 using the MMP-28 gene, also located on chromosome 17 as a control. By quantifying the number of copies of the HER2/neu gene on chromosome 17, the kPCR assay of the present invention is a useful diagnostic tool to determine if a patient is a candidate for anti-HER2/neu gene therapy.

### BACKGROUND OF THE INVENTION

Breast cancer is the most frequent malignancy among women in western countries; it has an incidence rate in the United States of 111 cases per 100,000 woman-years and a mortality rate of 24 deaths per 100,000 woman-years. There are an estimated one million new cases of breast cancer diagnosed annually in the world. In breast cancer, the predominant genetic mechanism for oncogene activation is through gene amplification. The HER2/neu oncogene is the most frequently amplified oncogene in breast cancer and overexpression of the HER2/neu protein is associated with poor clinical outcome.

HER2/neu protein overexpression in breast cancer is mainly caused by HER2/neu gene amplification on chromosome 17. Approximately 20% to 35% of all breast cancers are reported to have HER2/neu gene amplifications. A number of clinical studies have demonstrated a link between HER2/neu gene amplification status and responsiveness or resistance to anti-HER2/neu therapy. Laboratory assessment of HER-2/neu status has become a critical step in determining the patient's eligibility for anti-HER2/neu therapy with trastuzumab, an antineoplastic monoclonal antibody (HERCEPTIN®, Genetech, South San Francisco, Calif.) that is directed specifically against the HER2/neu protein. HERCEPTIN® has been shown to improve outcomes for women with HER2/neu overexpressing metastatic breast cancer by inhibiting tumor cell growth and stimulating the patient's immune response against the tumors. In order to determine if a women is a good candidate for HERCEPTIN® treatment, methods to accurately detect HER2/neu protein overexpression or HER2/neu gene amplification in a specimen are necessary.

The HER2/neu gene is located on the long arm of chromosome 17 at loci 17q12-q21.32 and encodes a 185 kDa transmembrane glycoprotein, which belongs to the family of epidermal growth factor (“EGF”) receptor tyrosine kinases (“RTKs”). Järvinen and Liu, BREAST CANCER RESEARCH AND TREATMENT 78:299-311 (2003). Numerical or structural abnormalities of chromosome 17 are common in breast cancer; the most common being aneusomy (i.e., deviation from the normal state of disomy 17). Approximately 54% of invasive breast carcinomas display aneusomy 17 (i.e., either monosomy or polysomy) of chromosome 17. Three or more copies of chromosome 17 per cell confer a sufficiently aggressive phenotype to show significant correlation with high-grade carcinomas and metastases. Watters et al., BREAST CANCER RESEARCH AND TREATMENT 77: 109-113 (2003). While polysomy 17 is correlated with multiple copies of the HER2/neu gene due to an increased number of chromosome 17, it is not correlated with HER2/neu gene amplification; thus, patients with polysomy 17 would not receive any benefit from HERCEPTIN® therapy because the total gene copy number per chromosome remains normal. An accurate measure of the number of HER2/neu gene copies and/or HER2/neu protein overexpression is consequently critical when determining a women's candidacy for HERCEPTIN® therapy.

Currently used diagnostic tests to detect HER2/neu protein overexpression and gene amplification, respectively, include the immunohistochemisty (“IHC”) HERCEPTEST® (Genentech, South San Francisco, Calif.), which measures HER2/neu protein in the cell membrane using monoclonal or polyclonal antibodies against HER2/neu protein, and fluorescent in situ hybridization (“FISH”), which evaluates HER2/neu gene amplification using fluorescently labeled HER2/neu genomic DNA; both the IHC and FISH assays are approved by the United States Food and Drug Administration. With IHC, samples are measured on a scoring system where samples having staining scores of 0 and 1+ are classified as negative for HER2/neu protein overexpression, samples having staining scores of 2+ are classified as weakly positive, and samples having staining scores of 3+ are classified as strongly positive for HER2/neu overexpression. With FISH, gene amplification is determined by calculating the ratio of the number of gene copies to the number of chromosome copies; a ratio of higher than 2.0 indicates HER2/neu gene amplification. As previously noted, the number of HER2/neu gene copies is determined by labeling genomic DNA samples. The number of chromosome copies is most commonly determined by labeling the chromosome 17 centromere (“CEP17”).

As alternatives to IHC and FISH, quantitative PCR techniques have been described as alternative methods to detect HER2/neu gene amplification. In 2003, Königshoff et al. (CLINICAL CHEMISTRY 49(2):219-229 (2003)) described a real time PCR assay (i.e., a kPCR assay) for determining HER2/neu gene amplification. In the real time PCR assay of Königshoff et al., the primer/probes are designed from within the exon 2/intron 2 sequence of HER2/neu (GenBank Accession No. M12036). Königshoff et al. used IGF-1 located on chromosomal 12 at region 12q22 for the reference gene. Königshoff et al. explains that IGF-1 was chosen because it is located on a chromosome, i.e., chromosome 12, a gene least frequently numerically altered in breast tumors. Under this assay, HER2/neu gene amplification is calculated from the ratio of the determined gene copy numbers of HER2/neu and IGF-1 measured in separate PCRs. To simulate HER2/neu gene amplification in the tumor sample, DNA samples for HER2/neu determination are used in different concentrations (5000, 2500, 500, and 250 copies per PCR) and are compared with DNA samples for IGF-1 that are of constant concentration (always 500 per copy). The ratio of HER2/neu to IGF-1 for normal samples is calculated from two independent reactions containing 500 copies each.

### SUMMARY OF THE INVENTION

The kPCR assay of the present invention improves upon currently known methods in the art to determine if a woman is a candidate for anti-HER2/neu gene therapy with HERCEPTIN® or another comparable drug by providing a kPCR assay that accurately and independently quantifies the number of HER2/neu gene copies in human tissue. When compared against diagnostic methods currently used in the art for determining HER2/neu protein overexpression, the present invention is both cost and time effective. The present invention also improves upon the HER2/neu kPCR assay known in the art by using a reference gene that is located on the same chromosome as HER2/neu, i.e., chromosome 17. By using a control gene on the same chromosome as HER2/neu, the present invention increases the accuracy for determining precise copy number of HER2/neu that is located on chromosome 17. Further, through the selection of a control gene on chromosome 17, the kPCR assay of the present invention may be used to quantify additional genes on chromosome 17, such as for example, the tumor suppressor genes p53 and BRCA1 and the topoisomerase 88 alpha gene at 17q12-17q21.

In one embodiment of the invention, there is provided a method of quantifying genes on chromosome 17 comprising the steps of (a) selecting a target gene for identification on chromosome 17; (b) preparing primers and probes directed to the target gene; (c) quantifying the target gene using kinetic PCR to obtain a gene copy number for the target gene; and comparing the gene copy number of the target gene against a gene copy number obtained for a control gene also located on chromosome 17.

In another embodiment of the invention, there is provided a method of determining if a breast cancer patient is a candidate for anti-HER2/neu gene therapy comprising the steps of (a) quantifying HER2/neu on chromosome 17 by obtaining a gene copy number for HER2/neu; (b) quantifying MMP-28 as a chromosome 17 control by obtaining a gene copy number for MMP-28; and (c) comparing the gene copy number of HER2/neu to the gene copy number of MMP-28 by obtaining a ratio of HER2/neu gene copies to MMP-28 gene copies, wherein a breast cancer patient is a candidate for anti-HER2/neu gene therapy where the ratio of HER2/neu gene copies to MMP-28 gene copies is greater than 2. The HER2/neu gene copy number of step (a) may be further used to determine whether or not the patient will respond to anti-HER2/neu gene therapy, with a low HER2/neu copy number indicating that the patient is a low responder who will not respond well to anti-HER2/neu gene therapy and a high HER2/neu copy number indicating that the patient is a high responder who will respond well to anti-HER2/neu gene therapy. The HER2/neu gene copy number of a candidate patient who is found to be a good responder may be further used to determine therapeutic dosages of the anti-HER2/neu agent to be administered to the patient.

In a further embodiment of the invention, there is provided a target amplification assay for determining gene copy number of at least one target gene located on chromosome 17 comprising using kinetic PCR to independently determine the gene copy number of the at least one target gene and a control gene, wherein both the at least one target gene and the control gene are located on chromosome 17. The target amplification assay of the present invention may be used to determine the gene copy number of a single target gene in singleplex format or of multiple target genes in a multiplex format.

Additional aspects, advantages, and features of the invention will be set forth in part in the description that follows and other aspects, advantages, and features of the invention will become apparent to those skilled in the art upon examination of the following, or may be learned by practice of the invention.

### BRIEF DESCRIPTION OF THE DRAWINGS

FIGS. 1a to 1c show graphs of cycle threshold (Ct) versus DNA concentration (in log ng) for the HCC1954, MCF7, and HUT-78 cell lines that have undergone the kPCR assay of the present invention in both singleplex and multiplex formats.

FIG. 2 shows an agarose gel of amplicons of DNA extracted from four human breast cancer tissue samples (ILS tissue samples 1019, 1020, 1022, and 1024) and the HUT-78 cell line using kPCR assay of the present invention in singleplex and multiplex format.

FIG. 3 shows a comparative analysis of the quantification of HER2/neu in three human breast cancer tissue samples from three different sources (Biogenic breast carcinoma tissue sample 17(A); Aster and infiltrating ducal cancer tissue sample C2; and ILS infiltrating ducal carcinoma tissue sample 113) by IHC, FISH, and the kPCR assay of the present invention.

### DETAILED DESCRIPTION OF THE INVENTION

The following detailed description of the invention will describe the present invention with reference to specific embodiments of the invention. It is to be understood that the specific embodiments as described below are meant only to be illustrative and not to be limiting. Further, the terminology used herein is used for the purpose of describing particular embodiments of the invention and is not intended to be limiting. As used in this specification and the appended claims, the singular forms “a,” “an,” and “the” include plural referents unless the context clearly dictates otherwise.

The term “target” refers to a molecule, gene, or genome containing a nucleic acid sequence or sequence segment that is intended to be characterized by way of identification, quantification, or amplification.

The term “gene” refers to a particular nucleic acid sequence within a DNA molecule that occupies a precise locus on a chromosome and provides the coded instructions for synthesis of RNA, which, when translated into protein, leads to the expression of hereditary character. The term “genome” refers to a complete set of genes in the chromosomes of each cell of a specific organism.

The term “gene amplification” refers to an increase in the number of copies of a specific gene in an organism's genome. It is understood by one of ordinary skill in the art that the presence of multiple copies of a gene within a genome may result in the production of a corresponding protein at elevated levels.

As used herein, the term “nucleic acid” refers to polynucleotide such as deoxyribonucleic acid (DNA), and, where appropriate, ribonucleic acid (RNA). The term should also be understood to include, as equivalents, analogs of RNA or DNA made from nucleotide analogs, and, as applicable to the embodiment being described, single (sense or antisense) and double-stranded polynucleotide. Chromosomes, cDNAs, mRNAs, and rRNAs are representative examples of molecules that may be referred to as nucleic acids.

As used herein, the term “oligonucleotide” encompasses polydeoxyribonucleotides (containing 2-deoxy-D-ribose), polyribonucleotides (containing D-ribose), any other type of polynucleotide that is an N-glycoside of a purine or pyrimidine base, and other polymers containing normucleotidic backbones (e.g., protein nucleic acids and synthetic sequence-specific nucleic acid polymers commercially available from the Anti-Gene Development Group, Corvallis, Oreg., as NEUGENE™ polymers) or nonstandard linkages, providing that the polymers contain nucleobases in a configuration that allows for base pairing and base stacking, such as is found in DNA and RNA. Thus, “oligonucleotides” herein include double- and single-stranded DNA, as well as double- and single-stranded RNA and DNA:RNA hybrids, and also include known types of modified oligonucleotides, such as, for example, oligonucleotides wherein one or more of the naturally occurring nucleotides is substituted with an analog; oligonucleotides containing internucleotide modifications such as, for example, those with uncharged linkages (e.g., methyl phosphonates, phosphotriesters, phosphoramidates, carbamates, etc.), negatively charged linkages (e.g., phosphorothioates, phosphorodithioates, etc.), and positively charged linkages (e.g., aminoalkylphosphoramidates, aminoalkylphosphotriesters), those containing pendant moietics, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), and those containing alkylators. There is no intended distinction in length between the terms “polynucleotide” and “oligonucleotide,” and these terms will be used interchangeably. These terms refer only to the primary structure of the molecule. As used herein the symbols for nucleotides and polynucleotide are according to the IUPAC-IUBMB Joint Commission on Biochemical Nomenclature (see, http://www.chem.qmul.ac.uk/iupac/jcbn).

Oligonucleotides can be synthesized by known methods. Background references that relate generally to methods for synthesizing oligonucleotides include those related to 5′-to-3′ syntheses based on the use of β-cyanoethyl phosphate protecting groups. See, e.g., de Napoli et al., GAZZ CHIM ITAL 114:65 (1984); Rosenthal et al., TETRAHEDRON LETT 24:1691 (1983); Belagaje and Brush, NUC ACIDS RES 10:6295 (1977); in references which describe solution-phase 5′-to-3′ syntheses include Hayatsu and Khorana, J AM CHEM SOC 89:3880 (1957); Gait and Sheppard, NUC ACIDS RES 4: 1135 (1977); Cramer and Koster, ANGEW CHEM INT ED ENGL 7:473 (1968); and Blackburn et al., J CHEM SOC PART C, at 2438 (1967). Additionally, Matteucci and Caruthers, J AM CHEM SOC 103:3185-91 (1981) describes the use of phosphochloridites in the preparation of oligonucleotides; Beaucage and Caruthers, TETRAHEDRON LETT 22:1859-62 (1981), and U.S. Pat. No. 4,415,732 to Caruthers et al. describe the use of phosphoramidites for the preparation of oligonucleotides. Smith, AM BIOTECH LAB, pp. 15-24 (December 1983) describes automated solid-phase oligodeoxyribonucleotide synthesis; and T. Horn and M. S. Urdea, DNA 5:421-25 (1986) describe phosphorylation of solid-supported DNA fragments using bis(cyanoethoxy)-N,N-diisopropylaminophosphine. See also, references cited in Smith, supra; Warner et al., DNA 3:401-11 (1984); and T. Horn and M. S. Urdea, TETRAHEDRON LETT 27:4705-08 (1986).

As used herein, the term “probe” refers to an oligonucleotide that forms a hybrid structure with a target sequence contained in a molecule (i.e., a “target molecule”) in a sample undergoing analysis, due to complementarity of at least one sequence in the probe. Generally, the probe and target sequence complementarity will be in sense-anti-sense configuration. The nucleotides of any particular probe may be deoxyribonucleotides, ribonucleotides, and/or synthetic nucleotide analogs.

The term “primer” refers to an oligonucleotide, whether produced naturally as in a purified restriction digestion or produced synthetically, which is capable of acting as a point of initiation of synthesis when placed under conditions in which synthesis of a primer extension product that is complementary to a nucleic acid strand is induced, i.e., in the presence of appropriate nucleotides and an agent for polymerization such as a DNA polymerase in an appropriate buffer and at a suitable temperature.

The terms “nucleotide” and “nucleoside” refer to nucleosides and nucleotides containing not only the four natural DNA nucleotidic bases, i.e., the purine bases guanine (G) and adenine (A) and the pyrimidine bases cytosine (C) and thymine (T), but also the RNA purine base uracil (U), the non-natural nucleotide bases iso-G and iso-C, universal bases, degenerate bases, and other modified nucleotides and nucleosides. Universal bases are bases that exhibit the ability to replace any of the four normal bases without significantly affecting either melting behavior of the duplexes or the functional biochemical utility of the oligonucleotide. Examples of universal bases include 3-nitropyrrole and 4-, 5-, and 6-nitroindole, and 2-deoxyinosine (dI), that latter considered the only “natural” universal base. While dI can theoretically bind to all of the natural bases, it codes primarily as G. Degenerate bases consist of the pyrimidine derivative 6H,8H-3,4-dihydropyrimido[4,5-c][1,2]oxazin-7-one (P), which when introduced into oligonucleotides base pairs with either G or A, and the purine derivative N6-methoxy-2,6,-diaminopurine (K), which when introduced into oligonucleotides base pairs with either C or T. Examples of the P and K base pairs include P-imino, P-amino, K-imino, and K-amino.

Modifications to nucleotides and nucleosides include, but are not limited to, methylation or acylation of purine or pyrimidine moieties, substitution of a different heterocyclic ring structure for a pyrimidine ring or for one or both rings in the purine ring system, and protection of one or more functionalities, e.g., using a protecting group such as acetyl, difluoroacetyl, trifluoroacetyl, isobutyryl, benzoyl, and the like. Modified nucleosides and nucleotides also include modifications on the sugar moiety, e.g., wherein one or more of the hydroxyl groups are replaced with halide and/or hydrocarbyl substituents (typically aliphatic groups, in the latter case), or are functionalized as ethers, amines, or the like. Examples of modified nucleotides and nucleosides include, but are not limited to, 1-methyladenine, 2-methyladenine, N⁶-methyladenine, N′-isopentyl-adenine, 2-methylthio-N′-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromo-guanine, 8-chloroguanine, 8-aminoguanine, 8-methylguanine, 8-thioguanine, 5-fluoro-uracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methyl-aminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine, and 2,6-diaminopurine.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of 80% or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer. Such minor modifications of the disclosed sequences and any necessary adjustments of hybridization conditions to maintain specificity require only routine experimentation and are within the ordinary skill in the art.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation. Typical hybridizing conditions include the use of solutions buffered to a pH from about 7 to about 8.5 and temperatures of from about 30° C. to about 60° C. Hybridization conditions also include a buffer that is compatible, i.e., chemically inert, with respect to primers, probes, and other components, yet still allows for hybridization between complementary base pairs, can be used. The selection of such buffers is within the knowledge of one of ordinary skill in the art.

It is understood by one of ordinary skill in the art that the isolation of DNA and RNA target sequences from a sample requires different conditions. For example, if the sample is initially disrupted in an alkaline buffer, double stranded DNA is denatured and RNA is destroyed. By contrast, if the sample is harvested in a neutral buffer with SDS and proteinase K, DNA remains double stranded and cannot hybridize with the primers and/or probes and the RNA is protected from degradation.

As used herein, the term “target amplification” refers to enzyme-mediated procedures that are capable of producing billions of copies of nucleic acid target. Examples of enzyme-mediated target amplification procedures known in the art include PCR, nucleic acid-sequence-based amplification (“NASBA”), transcription-mediated amplification (“TMA”), strand displacement amplification (“SDA”), and ligase chain reaction (“LCR”).

The most widely used target amplification procedure is PCR, first described for the amplification of DNA by Mullins et al. in U.S. Pat. No. 4,683,195 and Mullis in U.S. Pat. No. 4,683,202. The PCR procedure is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.”

In the PCR technique, a sample of DNA is mixed in a solution with a molar excess of two oligonucleotide primers of 10-30 base pairs each that are prepared to be complementary to the 3′ end of each strand of the DNA duplex; a molar excess of unattached nucleotide bases (i.e., dNTPs); and DNA polymerase, (preferably Taq polymerase, which is stable to heat), which catalyzes the formation of DNA from the oligonucleotide primers and dNTPs. Of the two primers, one is a forward primer that will bind in the 5′-3′ direction to the 3′ end of one strand of the denatured DNA analyte and the other is a reverse primer that will bind in the 3′-5′ direction to the 5′ end of the other strand of the denatured DNA analyte. The solution is heated to 94-96° C. to denature the double-stranded DNA to single-stranded DNA. When the solution cools, the primers bind to the separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. In other words, an extension product synthesized from the forward primer, upon separation, would serve as a template for a complementary extension product synthesized from the reverse primer. Similarly, the extension product synthesized from the reverse primer, upon separation, would serve as a template for a complementary extension product synthesized from the forward primer. In this way, the region of DNA between the primers is selectively replicated with each repetition of the process. Since the sequence being amplified doubles after each cycle, a theoretical amplification of one billion copies may be attained after repeating the process for a few hours; accordingly, extremely small quantities of DNA may be amplified using PCR in a relatively short period of time.

Because the amount of DNA theoretically doubles with every cycle of PCR, after each cycle, the amount of DNA is twice what it was before, consequently, after two cycles there is 2×2 (2²) or four times as much DNA; after three cycles there is 2×2×2 (2³) or eight times as much DNA; and after four cycles there is 2×2×2×2 (2⁴) or 16 times as much DNA; thus, after N cycles there is 2^{N} times as much DNA; the reaction eventually reaches a plateau phase at which time no further amplification proceeds. As a result of this type of amplification, PCR graphs that plot PCR cycle number (x-axis) versus amount of DNA (y-axis) begin linearly and curve exponentially. Because PCR is a logarithmic reaction, in order to see the amplification at the early stages, it is preferred to analyze DNA amplification from PCR on a logarithmic scale.

Where the starting material for the PCR reaction is RNA, complementary DNA (“cDNA”) is made from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. The enzymes are used in genetic engineering to produce specific cDNA molecules from purified preparations of mRNA. A PCR used to amplify RNA products is referred to as reverse transcriptase PCR or “RT-PCR.”

The terms “kinetic PCR” (“kPCR”) or “kinetic RT-PCR” (“kRT-PCR”), which are also referred to as “real-time PCR” and “real-time RT-PCR,” is a PCR that allows for the study of PCR reaction products at the early stages of amplification (i.e., at the linear range on a normal scale). Kinetic PCRs detect PCR products via a fluorescent signal generated by the coupling of a fluorogenic dye molecule and a quencher moiety to the same or different oligonucleotide substrates. Because the dye binds to amplified DNA products, a measure of the increase in fluorescence is equal to a measure of the increase in DNA product since the dye binds to the increasing amount of DNA in the reaction tube. In kPCR reactions, the measure of DNA or cDNA is determined logarithmically as discussed above. The threshold of the log graph is that point at which the linear graph starts to upturn as a result of the amplification. On the log graph, the point at which the fluorescence crosses the threshold is the threshold cycle or C_{T}. The threshold cycle or C_{T} value reflects the cycle number at which florescence generated within a reaction cross the threshold.

Examples of commonly used probes used in kPCR and kRT-PCR include the following probes: TAQMAN® probes, Molecular Beacons probes, SCORPION® probes, and SYBR® Green probes. Briefly, TAQMAN® probes, Molecular Beacons, and SCORPIONS probes each have a fluorescent reporter dye (also called a “fluor”) attached to the 5′ end of the probes and a quencher moiety coupled to the 3′ end of the probes. In the unhybridized state, the proximity of the fluor and the quench molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR® Green probes binds double-stranded DNA and upon excitation emit light; thus as PCR product accumulates, fluorescence increases.

The term “singleplex” refers to a single assay that is not carried out simultaneously with any other assays. Singleplex assays include individual assays that are carried out sequentially. Within the context of the present invention, when a kPCR assay is used to detect the copy number of a single gene, it is being used in singleplex format.

The term “multiplex” refers to multiple assays that are carried out simultaneously, in which detection and analysis steps are generally performed in parallel in a single reaction vessel, such as a tube or a well of a reaction plate. As used herein, a multiplex assay may also be termed according to the number of genes that the assay aims to identify. For example, using the kPCR assay described herein, a multiplex assay may detect the gene copy number of two or more genes on chromosome 17. In one embodiment described herein, the kPCR assay of the present invention is used to multiplex HER2/neu and MMP-28 gene copy number per cell (see, Examples 2 and 3).

The following description of the preferred embodiments and examples are provided by way of explanation and illustration and are not to be viewed as limiting the scope of the invention as defined by the claims. Further, when examples are given, they are intended to be exemplary only and not to be restrictive.

The kPCR assay of the present invention accurately determines the number of copies of the HER2/neu gene in human cells by quantifying the number of HER2/neu gene copies on chromosome 17 relative to a reference gene on the same chromosome that has a normal gene copy. With this procedure, true HER2/neu gene amplification on chromosome 17 can be distinguished from cases of chromosome 17 aneuploidy. The reference gene that is used in the HER2/neu gene assay of the present invention is the matrix metalloproteinase 28 gene (“MMP-28”). Matrix metalloproteinases (“MMPs”) are a comprehensive family of zinc metalloenzymes that are involved in the breakdown of extracellular matrix proteins; MMP-28 is located on chromosome 17 at the 17q11.2-17q12 loci. Marchenko and Strongin, GENE 265:87-93 (2001). Example 1 shows HER2/neu and MMP-28 sequences that are used in the kPCR assay of the present invention. The MMP-28 gene is a reliable gene to use as a reference because there are no reports of abnormal MMP-28 gene copy number in breast cancer patients. While there have been reports of MMP-28 overexpression in some cancers, because the kPCR assay is premised upon detecting overamplification not overexpression, any overexpression of MMP-28 will not effect the ability of the HER2/neu kPCR assay of the present invention to detect HER2/neu gene copy number.

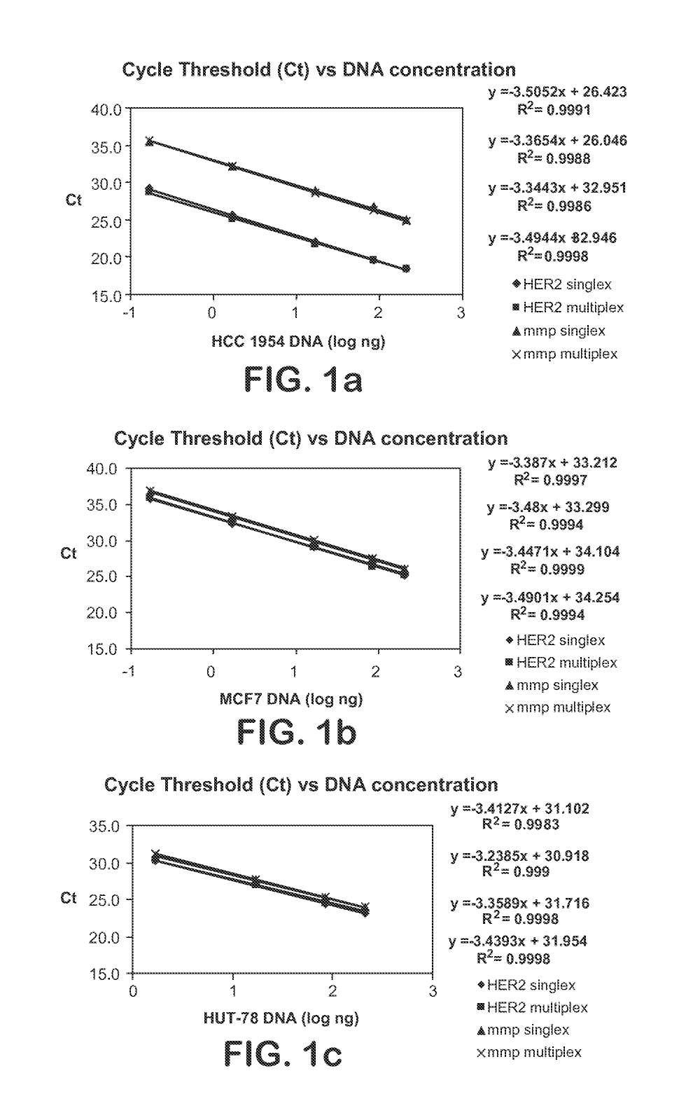

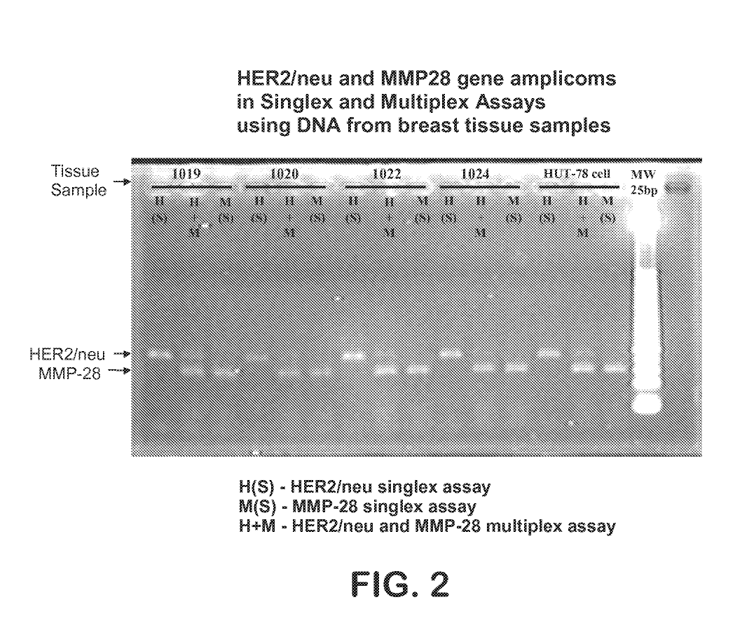

The chromosome 17 kPCR of the present invention may be used to perform multiplex assays that simultaneously detect the HER2/neu gene and the MMP-28 reference gene. As shown in FIGS. 1a-1c, FIG. 2, and Table 1 (Example 2), the kPCR assay of the present invention has identical accuracy in both singleplex and multiplex format. Because the multiplex assay allows for the simultaneous testing of a chromosome 17 gene, such as HER2/neu, along with a reference gene of known copy number, such as MMP-28, the copy number of the HER2/neu gene may be quantified by comparing the results of the assay for HER2/neu against that of the MMP-28 reference gene without the need of a standard curve for HER2/neu or MMP-28 gene (Example 3).

With its high degree of specificity for the detection of HER2/neu gene copy number in small tissue samples, the kPCR assay of the present invention is a useful diagnostic tool for the detection of the HER2/neu gene in breast tissue samples. As previously discussed, the identification of the HER2/neu gene in breast cancer patients is essential in order to determine if the patient is a candidate for anti-HER2/neu gene therapy with HERCEPTIN® or a comparable drug. The accuracy of the kPCR assay is controlled by running the samples against the MMP-28 gene, as both HER2/neu and MMP-28 are located on chromosome 17. The kPCR assay of the present invention has the advantage of being equally effective in both singleplex and multiplex format, thus enhancing the flexibility, accuracy, and cost effectiveness of the assay.

In view of the foregoing, in one embodiment, the present invention is directed to a method of quantifying genes on chromosome 17 comprising the steps of (a) selecting a target gene for identification on chromosome 17; (b) preparing printers and probes directed to the target gene; (c) quantifying the target gene using kinetic PCR to obtain a gene copy number for the target gene; and (d) comparing the gene copy number of the target gene against a gene copy number obtained for a control gene also located on chromosome 17. As discussed herein, the target gene may be HER2/neu and the control gene may be MMP-28, both of which are located on chromosome 17. Primers and probes that may be used to amplify and detect the HER2/neu target gene may be selected from SEQ ID NOs. 1, 2, and 3. Primers and probes that may be used to amplify and detect the MMP-28 control gene may be selected from SEQ ID NOs. 4, 5, and 6.

In another embodiment, the present invention is directed to a method of determining if a breast cancer patient is a candidate for anti-HER2/neu gene therapy comprising the steps of (a) quantifying HER2/neu on chromosome 17 by obtaining a gene copy number for HER2/neu; (b) quantifying MMP-28 as a chromosome 17 control by obtaining a gene copy number for MMP-28; and (c) comparing the gene copy number of HER2/neu to the gene copy number of MMP-28 by obtaining a ratio of HER2/neu gene copies to MMP-28 gene copies, wherein a breast cancer patient is a candidate for anti-HER2/neu gene therapy where the ratio of HER2/neu gene copies to MMP-28 gene copies is greater than 2. The gene copy number of the HER2/neu target gene may be quantified using primers and probes of SEQ ID NOs. 1,2, and 3 and the gene copy number of the MMP-28 control gene may be quantified using primers and probes of SEQ ID NOs. 4, 5, and 6.

Because one of ordinary skill in the art to which the invention pertains will understand that there is a difference between candidacy for gene therapy treatment and efficacy, the present invention provides a method for additionally determining whether or not a patient will respond to anti-HER2/gene therapy. For example, a patient with a low HER2/neu gene copy number may be considered a low responder who will not respond to anti-HER2/neu gene therapy while a patient with a high HER2/neu gene copy number may be considered a high responder who will respond well to anti-HER2/neu gene therapy. Midlevel responders may also be identified using this technique. The HER2/neu gene copy number of a candidate patient who is found to be a good responder may be further used to determine therapeutic dosages of the anti-HER2/neu agent to be administered to the patient.

In a further embodiment, the present invention is directed to a target amplification assay for determining gene copy number of at least one target gene located on chromosome 17 comprising using kinetic PCR to determine the gene copy number of the at least one target gene and a control gene, wherein both the at least one target gene and the control gene are located on chromosome 17. An example of a chromosome 17 target gene is HER2/neu and an example of a chromosome 17 control gene is MMP-28. As previously noted, the HER2/neu target gene may be amplified and detected with primers and probes selected from SEQ ID NOs. 1, 2, and 3 and the MMP-28 control gene may be amplified and detected with primers and probes selected from SEQ ID NOs. 4, 5, and 6.

While the kPCR assay of the present invention and related methods have been described herein for the detection and quantification of HER2/neu using MMP-28 as a control, it is to be understood that the assay and its related methods are not limited to the detection of HER2/neu as a target gene and the kPCR assay of the present invention and its related methods can be used for the detection of any genes found on chromosome 17. For example, the gene copy number of the topoisomerase II alpha gene at the 17q12-q21 loci, which is close to the HER2/neu gene loci, may be determined using the kPCR assay of the present invention in a singleplex format or gene copy numbers of both HER2/neu and topoisomerase II alpha may be determined using the kPCR assay of the present invention in a multiplex format.

All patents and publications mentioned herein are hereby incorporated by reference in their entireties.

The following examples are put forth so as to provide those of ordinary skill in the art with a complete disclosure and description of how to make and use the compositions of the invention. The examples are intended as non-limiting examples of the invention. Efforts have been made to ensure accuracy with respect to numbers (e.g., amounts, temperature, etc.) but some experimental error and deviations should, of course, be allowed for. Unless indicated otherwise, parts are parts by weight, temperature is degrees centigrade and pressure is at or near atmospheric. All components were obtained commercially unless otherwise indicated.

### EXPERIMENTAL

The practice of the present invention will use, unless otherwise indicated, conventional techniques of molecular biology, biochemistry, microbiology, and the like, which are within the skill of the art. Such techniques are explained fully in the literature. See, for example, Sambrook, et al., MOLECULAR CLONING: A LABORATORY MANUAL, 2^{nd} ed. (1989); OLIGONUCLEOTIDE SYNTHESIS (M. J. Gait, ed., 1984); THE PRACTICE OF PEPTIDE SYNTHESIS, 2^{nd} ed. (M. Bodanszky and A. Bodanszky, Springer-Verlag, New York, N.Y., 1994); NUCLEIC ACID HYBRIDIZATION (B. D. Haines & S. J. Higgins, eds., 1984); and METHODS IN ENZYMOLOGY (Elsevier, Inc., Burlington, Mass.).

In the examples that follow, efforts have been made to ensure accuracy with respect to numbers (e.g., amounts, temperature, etc.), but experimental error and standard deviations should be taken into account when conducting the described experiments. Unless indicated otherwise, parts are parts by weight, temperature is degrees centigrade and pressure is at or near atmospheric. Unless otherwise indicated, all formulations described herein were performed with commercially available products.

### Example 1

### kPCR HER2/NEU and MMP-28 Assay Design and Performance

HER2/neu primer and probe DNA sequences:

Forward primer:

(SEQ ID NO.1)

5′ CCAGCTTGGCCCTTTCCT 3′
Reverse primer:

(SEQ ID NO.2)

5′ GAATGGGTCGCTTTTGTTCTTAG 3′
Probe:

(SEQ ID NO.3)

5′ R-TTCCCTAAGGCTTTCAGTACCCAGGATCTG-Q3′

MMP-28 primer and probe DNA sequences:

| | |
|---|---|
| Forward primer: | |
| 5′ AATTCGAGACCATTTTGCAAGAC 3′ | (SEQ ID NO.4) |
| Reverse primer: | |
| 5′ TGACACCGTTTTTCAAGAACTGA 3′ | (SEQ ID NO.5) |
| Probe: | |
| 5′ R-TGCCCTTCTCCTCAGGACCCCCT-Q 3′ | (SEQ ID NO.6) |

For the probes, R = reporter dye; Q = quenching
moiety.

Extraction and Purification of Genomic DNA: Genomic DNA from cell lines was extracted and purified using the QIAamp DNA Mini Kit (QIAGEN Inc., Valencia, Calif. and tissue samples were extracted using the procedures set forth in commonly owned U.S. Publication No. 2004/0018525 A1 to Wirtz et al., which is incorporated herein in its entirety.

kPCR Reaction Mix: TAQMAN® Buffer; primers/probe for HER2/neu; primers/probe for MMP-28; and 1 μL of DNA eluate in a total reaction volume of 25 μL (TAQMAN® Universal PCR Mastermix, Applied Biosystems, Foster City, Calif.).

Singleplex and Multiplex Assays: For the singleplex assay, primers and probes for only HER2/neu or MMP-28 were used and for the multiplex assays, primers and probes for both HER2/neu and MMP-28 were used.

PCR Thermal Cycle Conditions: step 1-50° C., 2 minutes; step 2-95° C., 10 minutes; step 3-95° C., 15 seconds; and step 4-60° C., 1 minute. Steps 3 and 4 are repeated for 40 cycles.

### Example 2

### Performance of the kPCR Assay In Singleplex and Multiplex Format to Detect HER2/NEU and MMP-28 in Established Cancer Cell Lines

The performance of the kPCR assay of the present invention was analyzed by conducting side-to-side singleplex and multiplex assays specific for HER2/neu and MMP-28 on the following three cell lines:

HCC1954 (human infiltrating ducal carcinoma; ATCC® No. CRL-2338P (Manassas, Va.));

MCR7 (human mammary gland adenocarcinoma; ATTC® No. HTB-22 (Manassas, Va.)); and

HUT 78 (human cutaneous T cell lymphoma, ATCC® No. TIB-161 (Manassas, Va.)).

Genomic DNA was extracted and purified from the cells lines as set forth in Example 1. The purity of the DNA was determined through optical density (OD) measurements. The gene ratio of HER2/neu to MM28 in the purified DNA was determined using the kPCR assay of the present invention with the primers, probes, and procedures set forth in Example 1 in both singleplex and multiplex format. The results of the assay for each cell line were plotted logarithmically as shown in FIGS. 1a-1c with DNA concentration (log ng) on the x-axis and cycle threshold (Ct) on the y-axis. Using the formula “Exponential amplification=10^{(-1 slope)},” linear regression through a plot of Ct at each dilution against the log of genomic DNA produces the average efficiency of the PCR reaction. See, Michael W. Pfaffi, NUCLEIC ACIDS RESEARCH 29(9) 2002-2007 (2001). In FIGS. 1a-1c, R² is the percentage determined by the fit and R² is the variance of predicted values versus the variance of actual values; with a best fit, R² is 0.99-1.

As shown in FIG. 1, the results of the HER2/neu and MMP-28 singleplex assays were identical to the results of the multiplex assay for HER2/neu and MMP-28.

### Example 3

### Performance and specificity of the kPCR Assay In Singleplex and Multiplex Format to Detect HER2/NEU and MMP-28 In Human Breast Tissue Samples

The performance of the kPCR assay of the present invention was analyzed by conducting side-to-side singleplex and multiplex assays specific for HER2/neu and MMP-28 on the following four human breast cancer tissue lines obtained from Integrated Laboratory Services (ILS, Research Triangle Park, N.C.): ILS 1019; ILS 1020; ILS 1022; and ILS 1024. The tissues were fixed by formalin or formaldehyde and paraffin embedded on slides.

Genomic DNA was extracted and purified from the tissue samples as set forth in Example 1. The Ct of the purified genomic DNA samples were analyzed with the kPCR assay of the present invention using the primers, probes, and procedures set forth in Example 1 in both singleplex and multiplex format. As shown in Table 1, the Ct for the HER2/Neu and the MMP-28 in the singleplex assays were almost identical to the Ct for the HER2/neu and MMP-28 in the multiplex assays.

TABLE 1

| | SINGLEPLEX | MULTIPLEX | | SINGLEPLEX |
|---|---|---|---|---|
| DNA | HER2/NEU (Ct) | HER2/NEU (Ct) | MMP-28 (Ct) | MMP-28 (Ct) |
| Tissue ILS 1019 | 26.76 | 26.96 | 27.97 | 27.80 |
| Tissue ILS 1020 | 34.16 | 34.16 | 32.82 | 32.83 |
| Tissue ILS 1022 | 28.90 | 29.13 | 28.24 | 28.22 |
| Tissue ILS 1024 | 29.31 | 29.63 | 28.57 | 28.60 |
| HUT-78 Cell | 23.53 | 23.53 | 23.75 | 23.67 |

The specificity of the kPCR assay of the present invention was determined by running the amplification products of Table 1 on an agarose gel; the results are shown in FIG. 2. The results of the gel run confirmed that the kPCR assay of the present invention was specific for the HER2/neu and MMP-28 genes when run against a 25 bp DNA ladder marker.

### Example 4

### Comparative Analysis of IHC, Fish, and the kPCR Assay on Human Breast Tissue Samples

Breast tissue samples were obtained from three commercial sources (ILS, Research Triangle Park, N.C.; Asterand, Detroit, Mich.; and Biogenic, San Ramon, Calif.); the samples are listed in Table 2. The kPCR assay of the present invention was used in a multiplex format with MMP-28 as the reference gene to quantify the HER2/neu gene copy number per cell in all cell lines with known copy number. The kPCR assay of the present invention correctly detected 16/20 (80%) of patients with known HER2/neu IHC status (samples 4, 11, 17, and 18 did not have kPCR results consistent with the IHC results). FISH reflex testing of two of the four discrepant samples (samples 17 and 18) showed 100% correlation, i.e., the results for the two samples were consistent with the kPCR results.

TABLE 2

| | | | | IHC | kPCR RESULT | | |
|---|---|---|---|---|---|---|---|
| | | | PATHOLOGICAL | RESULT | (HER2/NEU GENE | | |
| | | | DIAGNOSIS | HER2 | COPY/CELL) | | |
| | SAMPLE | | CAUSE OF | PROTEIN | SECTION | SECTION | |
| SOURCE | NO. | TISSUE | DEATH | LEVEL | 1 | 2 | FISH |
| ILS | 1 | 1017 | Infiltrating | 3+ | 6.66 | 9.85 | |
| | | | Ducal CA | | | | |
| | 2 | 1018 | Mucinous CA | 3+ | 18.00 | 18.90 | |
| | 3 | 1019 | Infiltrating | 3+ | 4.35 | 6.08 | |
| | | | Ducal CA | | | | |
| | 4 | 1024 | Infiltrating | 3+ | 0.99 | 0.80 | |
| | | | Ducal CA | | | | |
| | 5 | 11151 | Infiltrating | + | 5.43 | 5.22 | |
| | | | Ducal CA | | | | |
| | 6 | 11155 | Infiltrating | + | 8.51 | 5.50 | |
| | | | Ducal CA | | | | |
| | 7 | 11156 | Infiltrating | + | 5.64 | 3.86 | |
| | | | Ducal CA | | | | |
| | 8 | 1020 | Fibrocystic | | 0.33 | 0.33 | |
| | | | Change | | | | |
| | 9 | 1022 | MVA | | 1.04 | 1.07 | |
| | 10 | 1023 | Fibrocystic | | 0.74 | 1.20 | |
| | | | Change | | | | |
| | 11 | 11152 | Mixed Mucinous | | 6.17 | 4.71 | |
| | | | CA | | | | |
| | 12 | 11153 | Infiltrating | | 1.13 | 1.13 | |
| | | | Ducal CA | | | | |
| | 13 | 11154 | Infiltrating | | 0.55 | 0.54 | |
| | | | Ducal CA | | | | |
| | 14 | 11157 | Infiltrating | | 1.65 | 1.43 | |
| | | | Ducal CA | | | | |
| | 15 | 11158 | Infiltrating | | 1.03 | 0.76 | |
| | | | Ducal CA | | | | |
| | 16 | 11159 | NL | Neg | 0.92 | 0.85 | |
| Asterand | 17 | C2 | Infiltrating | 3+ | 1.35 | 1.40 | Low |
| | | | Ducal CA | | | | amplification |
| | | | | | | | or NL |
| Biogenic | 18 | 17(A) | Breast CA | High | 1.17 | 1.15 | Low |
| | | | | | | | amplification |
| | | | | | | | or NL |
| | 19 | 143 | Breast CA | High | 8.28 | | High |
| | | | | | | | amplification |
| | 20 | 691 | Breast CA | High | 7.81 | | Low |
| | | | | | | | amplification |
| | | | | | | | or NL |

CA = carcinoma
MVA = Motor Vehicle Accident
NL = Normal

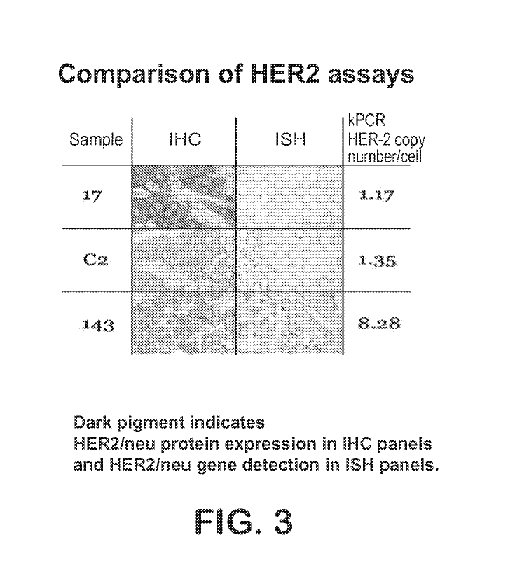

FIG. 3 shows a comparative analysis of samples 17 (Aster and C2), 18 (Biogenic 17(A)), and 19 (Biogenic 143) using IHC with anti-human HER2/neu oncoprotein (Dako Corp., Carpenteria, Calif.), FISH (HER2 kit, Zymed Laboratories, Inc. South San Francisco, Calif.), and the kPCR assay of the present invention. The dark pigment around the cells in the IHC samples are HER2/neu protein expression and the dark pigmented dots in the cells in the FISH samples are indicative of HER2 gene detection. Unlike IHC and FISH, where gene detection is determined by analyzing stained cells, the kPCR assay of the present invention is capable of quantifying gene copy number numerically. In FIG. 3, the gene copy number is calculated using ABI Prism 7700 Sequence Detection System (ABI User Bulletin #2) and the comparative C_{T} method described above.

We claim:

1. A method of quantifying genes on chromosome 17 comprising the steps of:
(a) selecting a target gene for identification on chromosome 17;
(b) preparing primers and probes directed to the target gene;
(c) quantifying the target gene using kinetic PCR (kPCR) to obtain a gene copy number for the target gene;
(d) comparing the gene copy number of the target gene against a gene copy number obtained for a control gene also located on chromosome 17.

2. The method of claim 1, wherein the at least one target gene is HER2/neu.

3. The method of claim 1, wherein the control gene is MMP-28.

4. The method of claim 2, wherein the HER2/neu target gene is amplified and detected using primers and probes selected from SEQ ID NOs. 1, 2, and 3.

5. The method of claim 3, wherein the MMP-28 control gene is amplified and detected using primers and probes selected from SEQ ID NOs. 4, 5, and 6.

6. A method of determining if a breast cancer patient is a candidate for anti-HER2/neu gene therapy comprising the steps of:
(a) quantifying target gene HER2/neu on chromosome 17 by obtaining a gene copy number for HER2/neu;
(b) quantifying control gene MMP-28 on chromosome 17 by obtaining a gene copy number for MMP-28; and
(c) comparing the gene copy number of HER2/neu to the gene copy number of MMP-28 by obtaining a ratio of HER2/neu gene copies to MMP-28 gene copies,
wherein a breast cancer patient is a candidate for anti-HER2/neu gene therapy where the ratio of HER2/neu gene copies to MMP-28 gene copies is greater than 2.

7. The method of claim 6, wherein the HER2/neu gene copy number of step (a) is used to determine whether or not the patient will respond to anti-HER2/neu gene therapy.

8. The method of claim 7, wherein a low copy number of HER2/neu indicates that the patient is a low responder who will not respond well to anti-HER2/neu gene therapy.

9. The method of claim 7, wherein a high copy number of HER2/neu indicates that the patient is a high responder who will respond well to anti-HER2/neu gene therapy.

10. The method of claim 9, wherein the HER2/neu gene copy number of step (a) is further used to determine therapeutic dosages of the anti-HER2/neu agents to be administered to the patient.

11. The method of claim 6, wherein the HER2/neu target gene is amplified and detected using primers and probes selected from SEQ ID NOs. 1, 2, and 3.

12. The method of claim 6, wherein the MMP-28 control gene is amplified and detected using primers and probes selected from SEQ ID NOs. 4, 5, and 6.

13. A target amplification assay for determining gene copy number of at least one target gene located on chromosome 17 comprising using kinetic PCR (kPCR) to independently determine the gene copy number of the target gene and a control gene, wherein both the target gene and the control gene are located on chromosome 17.

14. The assay of claim 13, wherein the at least one target gene is HER2/neu.

15. The assay of claim 13, wherein the control gene is MMP-28.

16. The assay of claim 14, wherein the HER2/neu target gene is amplified and detected using primers and probes selected from SEQ ID NOs. 1, 2, and 3.

17. The assay of claim 15, wherein the MMP-28 control gene is amplified and detected using primers and probes selected from SEQ ID NOs. 4, 5, and 6.

18. The assay of claim 14, wherein the assay is used to determine the gene copy number of HER2/neu and topoisomerase 88 alpha in a multiplex format.

19. The assay of claim 17, wherein the control gene is MMP-28.
